(19) United States
(12) Patent Application Publication
(10) Pub. No.: US 2012/0052121 A1
(43) Pub. Date: Mar. 1, 2012

(54) NEW COMBINATION OF ACTIVE INGREDIENTS CONTAINING A NON STEROIDAL ANTI INFLAMMATORY DRUG AND A COLCHICOSIDE DERIVATIVE

(75) Inventors: Praveen KHULLAR, Goa (IN); Mansing SHINGTE, Goa (IN); Shirishbhai PATEL, Goa (IN); Suseendharnath A, Goa (IN); Krishna RAJU, Goa (IN); Vanga REDDY, Goa (IN)

(73) Assignee: SANOFI, Paris (FR)

(21) Appl. No.: 13/188,772

(22) Filed: Jul. 22, 2011

Related U.S. Application Data
(63) Continuation of application No. PCT/IN2009/000071, filed on Jan. 22, 2009.

(51) Int. Cl. A61K 31/704; A61P 29/00; A61P 19/02; A61P 25/00; A61K 9/28; A61P 21/00

(52) U.S. Cl. 424/474; 514/33

(57) ABSTRACT

Pharmaceutical composition containing a combination of a non steroidal anti inflammatory drug and a colchicoside derivative, the active ingredients being present in the free state or in the form of a salt.

The subject of the present invention is a new combination of a non steroidal anti inflammatory drug and a colchicoside derivative; pharmaceutical compositions containing them for ameliorating and/or treating musculoskeletal and joint disorders such as ankylosing spondylitis, low back pain, osteoarthritis, and rheumatoid arthritis, and peri-articular disorders such as bursitis and tendonitis, and painful muscle spasm; and their manufacturing process.

Among those disorders, low back pain (LBP) is a very common painful musculoskeletal disorder that affects virtually everyone at some time during their life, and has a lifetime prevalence ranging from 58% to 84%. Low back problems rank high among the reasons for physician office visits and are costly in terms of medical treatment, lost productivity, and non monetary costs such as diminished ability to perform or enjoy usual activities. In fact, for people under age 45, low back problems are the most common cause of disability.

Among the non steroidal anti inflammatory drug known from the prior art that can be use in the instant invention, there is ketoprofen. Ketoprofen or (RS)-2-(3-benzoylphenyl)propanoic acid is a non steroidal anti inflammatory drug. The anti-inflammatory, analgesic, and antipyretic properties of ketoprofen have been demonstrated in classical animal and in vitro test systems. In anti-inflammatory models ketoprofen has been shown to have inhibitory effects on prostaglandin and leukotriene synthesis, to have antibradykinin activity, as well as to have lysosomal membrane-stabilizing action. Ketoprofen can be synthesized by methods known in the art such as in patent U.S. Pat. No. 3,641,127 or FR2163875.

Among the colchicoside derivative known from the prior art that can be use in the instant invention, there is thiocolchicoside. Thiocolchicoside or N-[1,2-dimethoxy-10-methylsulfanyl-9-oxo-3-[(2S,3R,4S,5S,6R)-3,4,5-trihydroxy-6-(hydroxymethyl)oxan-2-yl]oxy-6,7-dihydro-5H-benzo[d]heptalen-7-yl]acetamide is a glucosidal extracted from the seeds of Colchicum autumnale. It has muscle relaxant, anti-inflammatory, analgesic and anesthetic actions with minimal side effects. Thiocolchicoside can be synthesized by methods known in the art such as in patent FR1049755.

The active ingredients constituting the combination are present in the free state or in the form of one their salts.

These salts include for example salts with mineral acids such as hydrochloric acid, hydrobromic acid, sulfuric acid, nitric acid, phosphoric acid; salts with organic acids such as methanesulfonic acid, benzenesulfonic acid, p-toluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, and salicylic acid; or salts with acidic amino acids such as aspartic, and glutamic acid. Pharmacological acceptable salts are preferred.

Another object of the present invention is a pharmaceutical composition containing a combination of a non steroidal anti inflammatory drug and thiocolchicoside, both active ingredients being present in the free state or in the form of a salt.

Another object of the present invention is a pharmaceutical composition containing a combination of a ketoprofen and thiocolchicoside, both active ingredients being present in the free state or in the form of a salt.

Another object of the present invention is a pharmaceutical composition in a form being able to be administered by the oral route.

Another object of the present invention is a pharmaceutical composition in the form of a solid dosage form.

Another object of the present invention is a pharmaceutical composition in the form of a film coated tablet.

The present invention has the advantage to provide a stable combination product providing superior analgesic, anti inflammatory and muscle relaxant property compared to plain thiocolchicoside tablets. It furthers provides a combination product with reduced and controlled impurities.

The pharmaceutical composition and its formulation process involve avoiding chemical interaction of thiocolchicoside with ketoprofen, using pharmaceutically acceptable excipients in the dosage form. The impurity data profile reveals that when ketoprofen and thiocolchicoside are mixed in intimate contact with each other using pharmaceutically acceptable excipients, there is a significant increase in the level of degradation products as compared to, when they are separated in the dosage form. An object of the invention is a pharmaceutical composition containing ketoprofen and thiocolchicoside, being present in the free state or in the form of a salt, and not being intimately mixed in the composition.

Moreover, the combination product shows improved and controlled impurities even lesser than when compared to thiocolchicoside tablets of same dose when subjected to stress studies.

According to a preferred embodiment of the invention, the active ingredients of the combination are ketoprofen and thiocolchicoside. The usual oral dose for ketoprofen is 50 to 100 mg twice daily. The usual initial oral dose for thiocolchicoside is 16 mg daily.

In the pharmaceutical compositions of the present invention, the active ingredients are generally formulated in dosage units containing from 50 to 100 mg of ketoprofen and 4 to 8 mg of thiocolchicoside per unit dosage.

Another object of the present invention is a pharmaceutical composition containing 50 mg of ketoprofen and 4 mg of thiocolchicoside.

Another object of the present invention is a pharmaceutical composition containing 100 mg of ketoprofen and 8 mg of thiocolchicoside.

Another object of the present invention is a pharmaceutical composition containing 100 mg of ketoprofen and 8 mg of thiocolchicoside and in the form of a solid dosage form being divisible.

The dose and frequency of administration of the medicament of the present invention are not particularly limited, and they may be appropriately chosen depending on conditions such as the body weight or age of a patient, severity and the like. Generally, a daily dose for oral administration may be administered once a day or several times a day as divided portions, or once in several days.

According to another of its objects, the present invention relates to the use of a composition as previously described, for the preparation of a medicament intended for ameliorating and/or treating musculoskeletal and joint disorders such as ankylosing spondylitis, low back pain, osteoarthritis, and rheumatoid arthritis, and peri-articular disorders such as bursitis and tendonitis, and painful muscle spasm.

A further object of the present invention is a method for treating/ameliorating the pathologies indicated above, which comprises the administration to a patient of an effective amount of the composition according to the invention.

The pharmaceutical composition according to the present invention can further include others active ingredients having an acceptable pharmaceutical activity.

These compositions are preferably made so as to be administered by the oral or parenteral route, and more preferably by the oral route.

For example, the pharmaceutical composition may be formulated, for example, in the form of pharmaceutical compositions for oral administration such as granules, fine granules, powders, hard capsules, soft capsules, syrups, emulsions, suspensions, solutions and the like, or in the form for sublingual a buccal administration, or in the form of pharmaceutical compositions for parenteral administrations such as injections for intravenous, intramuscular, or subcutaneous administration, drip infusions, transdermal preparations, transmucosal preparations, nasal drops, inhalants, suppositories and the like. Injections or drip infusions may be prepared as powdery preparations such as in the form of lyophilized preparations, and may be used by dissolving just before use in an appropriate aqueous medium such as physiological saline. Sustained-release preparations such as those coated with a polymer may be directly administered intracerebrally. Preferably the pharmaceutical composition is in the form of a film coated tablet.

The tablets can be coated with sucrose or other appropriate materials or alternatively they can be treated such that they have a prolonged or delayed activity and that they have a prolonged or delayed activity and that they continuously liberate a predetermined quantity of active ingredient.

A preparation in the form of gelatin capsules is obtained by mixing the active ingredient with a diluent and by pouring the mixture obtained into soft or hard gelatin capsules. For the preparation of liquid compositions for oral administration, a conventional inert diluent such as water or a vegetable oil may be used. The liquid composition may contain, in addition to the inert diluent, auxiliaries such as moistening agents, suspension aids, sweeteners, aromatics, colorants, and preservatives. The liquid composition may be filled in capsules made of an absorbable material such as gelatin. Examples of solvents or suspension mediums used for the preparation of compositions for parenteral administration, e.g. injections, suppositories, include water, propylene glycol, polyethylene glycol, benzyl alcohol, ethyl oleate, lecithin and the like. Examples of base materials used for suppositories include, for example, cocoa butter, emulsified cacao butter, lauric lipid, witepsol.

A further object of the present invention is a method of making a tablet dosage form comprising the steps of

a) blending ketoprofen and pharmaceutically acceptable excipients to form a blended material
b) preparing a binder material with pharmaceutically acceptable excipients
c) adding the binder material to preparation containing ketoprofen
d) wet granulating the material obtained from c)
e) wet sizing
f) drying
g) dry sizing
h) blending thiocolchicoside and pharmaceutically acceptable excipients to form a blended material, all materials involved being sieved into a 1 mm sieve
i) blending material from step g) and h)
j) adding lubricant agent, all materials involved being sieved into a 1 mm sieve
k) tableting to form a tablet dosage form.

A further object of the present invention is a method of making a film coated tablet dosage form comprising the steps of making a tablet dosage form as previously described and after the step of heating and coating to form a film coated tablet dosage form. A further object of the present invention is a method of making a tablet dosage form containing ketoprofen and thiocolchicoside, wherein they are not intimately mixed.

The present invention is illustrated in the examples below which should not be interpreted as a limitation of the invention.

### EXAMPLE 1

### Manufacturing Process of the Tablet Containing Ketoprofen and Thiocolchicoside

Step 1: Sifted ketoprofen with pharmaceutical excipients and mixed.

Step 2: Prepared binder solution and granulated the material of step 1 to obtain uniform granules. The wet granules were dried to achieve optimum moisture required for compression.

Step 3: Sifted the dried granules and added thiocolchicoside, with pharmaceutical excipients and mixed.

Step 4: Sifted magnesium stearate and mixed.

Step 5: The blend was compressed into tablets using suitable tooling and coated using coating material.

Alternately tablets can also be prepared by dry granulation process such as the following

Step 1: Sifted ketoprofen, with pharmaceutical excipients and mixed.

Step 2: Compacted/slugged the contents and sieved.

Step 3: Sifted Thiocolchicoside and other pharmaceutical excipients and mixed.

Step 4: Sifted magnesium stearate and mixed.

Step 5: The blend was compressed into tablets and coated using coating material.

### EXAMPLE 2

### Film Coated Tablet Containing Ketoprofen 100 mg and Thiocolchicoside 8 mg

| | | QYT/ | QYT/ | QYT/ | QYT/ |
|---|---|---|---|---|---|
| | | TAB | TAB | TAB | TAB |
| No: | INGREDIENTS | (mg) | (mg) | (mg) | (mg) |
| 1. | Ketoprofen | 100.00 | 100.00 | 100.00 | 100.00 |
| 2. | Lactose Monohydrate | 367.60 | 340.50 | 321.75 | 253.00 |
| 3. | Croscarmellose Sodium | 1.00 | 5.00 | 7.50 | 21.00 |
| | (Ac di sol) | | | | |
| 4. | Maize starch | 10.00 | 25.00 | 34.50 | 62.50 |
| | BINDER SOLUTION | | | | |
| 5. | Povidone K-30 | 1.00 | 3.25 | 4.10 | 10.50 |
| 6. | Purified Water | qs | qs | qs | qs |
| | EXTRAGRANULAR | | | | |
| 7. | Croscarmellose Sodium | 1.00 | 3.50 | 6.75 | 20.00 |
| | (Ac di sol) | | | | |
| 8. | Colloidal silicone dioxide | 0.40 | 0.75 | 0.90 | 3.50 |
| 9. | Thiocolchicoside | 8.00 | 8.00 | 8.00 | 8.00 |
| 10. | Magnesium Stearate | 1.00 | 4.00 | 6.50 | 11.50 |
| | Tablet weight | 490.00 | 490.00 | 490.00 | 490.00 |
| 11. | COATING | | | | |
| 12. | Opadry yellow | qs | qs | qs | qs |
| 13. | Purified water | qs | qs | qs | qs |
| | Tablet weight | 500.00 | 500.00 | 500.00 | 500.00 |

### EXAMPLE 3

### Film Coated Tablet Containing Ketoprofen 50 mg and Thiocolchicoside 4 mg

| | | QYT/ | QYT/ | QYT/ | QYT/ |
|---|---|---|---|---|---|
| | | TAB | TAB | TAB | TAB |
| No: | INGREDIENTS | (mg) | (mg) | (mg) | (mg) |
| 1. | Ketoprofen | 50.000 | 50.000 | 50.000 | 50.000 |
| 2. | Lactose Monohydrate | 183.800 | 170.250 | 160.875 | 126.500 |
| 3. | Croscarmellose Sodium | 0.5000 | 2.500 | 3.750 | 10.500 |
| | (Ac di sol) | | | | |
| 4. | Maize starch | 5.000 | 12.500 | 17.250 | 31.250 |
| | BINDER SOLUTION | | | | |
| 5. | Povidone K-30 | 0.500 | 1.625 | 2.050 | 5.250 |
| 6. | Purified Water | qs | qs | qs | qs |
| | EXTRAGRANULAR | | | | |
| 7. | Croscarmellose Sodium | 0.500 | 1.750 | 3.375 | 10.000 |
| | (Ac di sol) | | | | |
| 8. | Colloidal silicone dioxide | 0.200 | 0.375 | 0.450 | 1.750 |
| 9. | Thiocolchicoside | 4.000 | 4.000 | 4.000 | 4.000 |
| 10. | Magnesium Stearate | 0.500 | 2.000 | 3.250 | 5.750 |
| | Tablet weight | 245.000 | 245.000 | 245.000 | 245.000 |
| 11. | COATING | | | | |
| 12. | Opadry yellow | qs | qs | qs | qs |
| 13. | Purified water | qs | qs | qs | qs |
| | Tablet weight | 250.000 | 250.000 | 250.000 | 250.000 |

### EXAMPLE 4

### Stability Comparative Data (40° C./Humidity Rate 75%)

| | A | B | C | D |
|---|---|---|---|---|
| | Lot 1 | Lot 2 | Lot 1 | Lot 2 |
| Impurities | Initial | Initial | 40° C./75% | 40° C./75% |
| Unknown impurity 1 | 0.25 | ND | 0.27 | ND |
| Unknown impurity 2 | 0.47 | ND | 0.26 | ND |
| Unknown impurity 3 | ND | ND | 1.17 | ND |
| Unknown impurity 4 | ND | ND | 1.14 | ND |
| Highest Unknown | 0.25 | 0.18 | 1.17 | 0.25 |
| Total impurities | 1.35 | 0.24 | 3.92 | 0.25 |

Column A: Lot 1 at initial time containing ketoprofen and thiocolchicoside prepared via combined granulation.
Column B: Lot 2 at initial time containing ketoprofen and thiocolchicoside prepared via extragranular granulation.
Column C: Lot 1 containing ketoprofen and thiocolchicoside prepared by combined granulation, after 2 months at 40° C./humidity rate 75% RH. The impurity data is tabulated representing 40° C./humidity rate 75% RH at 2M station.
Column D: Lot 2 containing ketoprofen and thiocolchicoside prepared by extragranular granulation, after 6 months at 40° C./humidity rate 75%, RH. The stability data shows that in Lot 1 (column A versus column C), there is significant increase in the level of impurities within 2 months at 40° C./humidity rate 75% RH in packed sample. The stability data shows that in Lot 2(column B versus column D), there is no significant change in the impurities even after 6 months at 40° C./humidity rate 75% RH packed sample. It is inferred from the above data that when ketoprofen and thiocolchicoside are not intimately mixed within the dosage form, there is no significant interaction and impurities are at substantially low level.

### EXAMPLE 5

### Safety and Efficacy of the Composition Containing Ketoprofen and Thiocolchicoside

The composition according to the invention can be considered as safe and efficient in acute LBP.

1. Pharmaceutical composition in the form of a solid dosage form containing a combination of active ingredients ketoprofen and thiocolchicoside, the active ingredients being present in the free state or in the form of a salt and not being intimately mixed in the composition.

2. Pharmaceutical composition according to claim 1, in a form being able to be administered by the oral route.

3. Pharmaceutical composition according to claim 1, in the form of a film coated tablet.

4. Pharmaceutical composition according to claim 1 containing 100 mg of ketoprofen and 8 mg of thiocolchicoside in the form of a solid dosage form being divisible.

5. Pharmaceutical composition according to claim 1, containing 50 to 100 mg of ketoprofen and 4 to 8 mg of thiocolchicoside.

6. Pharmaceutical composition according to claim 1, containing 100 mg of ketoprofen and 8 mg of thiocolchicoside.

7. Pharmaceutical composition according to claim 1, containing 50 mg of ketoprofen and 4 mg of thiocolchicoside.

8. Pharmaceutical composition according to claim 1, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

9. Use of a composition according to any one of the preceding claims, for the preparation of a medicament intended for ameliorating and/or treating musculoskeletal and joint disorders such as ankylosing spondylitis, low back pain, osteoarthritis, and rheumatoid arthritis, and peri-articular disorders such as bursitis and tendonitis, and painful muscle spasm.

10. A method of making a tablet dosage form comprising the steps of
a) blending ketoprofen and pharmaceutically acceptable excipients to form a blended material
b) preparing a binder material with pharmaceutically acceptable excipients
c) adding the binder material to preparation containing ketoprofen
d) wet-granulating the material obtained from c)
e) wet-sizing
f) drying
g) dry-sizing
h) blending thiocolchicoside and pharmaceutically acceptable excipients to form a blended material, all materials involved being sieved into a 1 mm sieve
i) blending material from step g) and h)
j) adding lubricant agent, all materials involved being sieved into a 1 mm sieve
k) tableting to form a tablet dosage form.

11. A method of making a film coated tablet dosage form comprising the steps of making a tablet dosage form according to claim 10 and after the step of heating and coating to form a film coated tablet dosage form.

12. Pharmaceutical composition according to claim 2, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

13. Pharmaceutical composition according to claim 3, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

14. Pharmaceutical composition according to claim 4, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

15. Pharmaceutical composition according to claim 5, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

16. Pharmaceutical composition according to claim 6, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.

17. Pharmaceutical composition according to claim 7, for the treatment or amelioration of musculoskeletal and joint disorders such as ankylosing spondylitis, low back pain, osteoarthritis, and rheumatoid arthritis, and peri-articular disorders such as bursitis and tendonitis, and painful muscle spasm.
